(19) United States
(12) Patent Application Publication
(10) Pub. No.: US 2025/0023390 A1
(43) Pub. Date: Jan. 16, 2025

(54) SYSTEMS AND METHODS FOR WIRELESS POWER RESONATORS WITH COUNTER-COIL

(71) Applicant: TC1 LLC, St. Paul, MN (US)

(72) Inventors: John Freddy Hansen, Livermore, CA (US); Russell Eugene Anderson, Hopkins, MN (US); Rachel Keen, Medford, MA (US); Daniel I. Harjes, Carlisle, MN (US); Jared Richard Johnson, Wyoming, MN (US)

(21) Appl. No.: 18/712,500

(22) PCT Filed: Nov. 29, 2022
(86) PCT No.: PCT/US2022/080545

§ 371 Date: May 22, 2024

Related U.S. Application Data
(60) Provisional application No. 63/285,637, filed on Dec. 3, 2021.

(51) Int. Cl. H02J 50/12; A61M 60/178; A61M 60/873; H01F 27/24; H01F 27/28; H01F 38/14; H02J 50/00

(52) U.S. Cl. CPC H02J 50/12; A61M 60/178; A61M 60/873; H01F 27/24; H01F 27/28; H01F 38/14; H02J 50/005; A61M 2207/00

(57) ABSTRACT

Systems and methods for a wireless power transfer system are provided. A resonator arrangement includes a housing, a magnetic core positioned within the housing and defining an annular groove, a coil element positioned within the annular groove and configured to generate a first magnetic field, and a counter-coil element positioned proximate the coil element, the counter-coil element configured to generate a second magnetic field that is out of phase with the first magnetic field to facilitate reducing far-field electromagnetic emissions.

### CROSS-REFERENCE TO RELATED APPLICATIONS

This application claims priority to provisional application Ser. No. 63/285,637, filed Dec. 3, 2021, which is incorporated herein by reference in its entirety.

### BACKGROUND OF THE DISCLOSURE

### a. Field of the Disclosure

The present disclosure relates generally to wireless power transfer systems, and more specifically, relates to wireless power transfer resonators including a counter-coil.

### b. Background

Ventricular assist devices, known as VADs, are implantable blood pumps used for both short-term (i.e., days or months) and long-term (i.e., years or a lifetime) applications where a patient's heart is incapable of providing adequate circulation, commonly referred to as heart failure or congestive heart failure. A patient suffering from heart failure may use a VAD while awaiting a heart transplant or as a long-term destination therapy. In another example, a patient may use a VAD while recovering from heart surgery. Thus, a VAD can supplement a weak heart (i.e., partial support) or can effectively replace the natural heart's function.

A wireless power transfer system may be used to supply power to the VAD. The wireless power transfer system generally includes an external transmit resonator (also referred to herein as a “transmitter (TX) module”) and an implantable receive resonator configured to be implanted inside a patient's body. This power transfer system may be referred to as a transcutaneous energy transfer system (TETS).

It is desirable to reduce far field electromagnetic (EM) emissions from a TETS. At a minimum, the TETS should comply with various standards, such as CISPR 11 group 2 Class B limit. The transmitter (TX) module in the TETS is generally the largest source of far-field emissions. Accordingly, a TX module that significantly reduces EM emissions (e.g., by at least 2 decibels (dB)) would be desirable.

### SUMMARY OF THE DISCLOSURE

In one aspect, a resonator arrangement for use in a wireless power transfer system is provided. The resonator arrangement includes a housing, a magnetic core positioned within the housing and defining an annular groove, a coil element positioned within the annular groove and configured to generate a first magnetic field, and a counter-coil element positioned proximate the coil element, the counter-coil element configured to generate a second magnetic field that is out of phase with the first magnetic field to facilitate reducing far-field electromagnetic emissions.

In another aspect, a transcutaneous energy transfer system (TETS) is provided. The TETS includes a transmit resonator including a first housing, a first magnetic core defining a first annular groove, and a transmit coil element positioned within the first annular groove and configured to generate a first magnetic field, the TETS further includes an implantable receive resonator including a second housing, a second magnetic core defining a second annular groove, and a receive coil element positioned within the second annular groove, wherein the first magnetic field is configured to induce a current in the receive coil element. The TETS further includes a counter-coil element positioned proximate the transmit coil element, the counter-coil element configured to generate a second magnetic field that is out of phase with the first magnetic field to facilitate reducing far-field electromagnetic emissions.

In yet another aspect, a method of assembling a resonator arrangement for use in a wireless power transfer system is provided. The method includes positioning a magnetic core within a housing, the magnetic core defining an annular groove, positioning a coil element within the annular groove, the coil element configured to generate a first magnetic field, and positioning a counter-coil element proximate the coil element, the counter-coil element configured to generate a second magnetic field that is out of phase with the first magnetic field to facilitate reducing far-field electromagnetic emissions.

### BRIEF DESCRIPTION OF THE DRAWINGS

FIG. 1 is a simplified electrical circuit diagram of a wireless power transfer system.

FIG. 2 is an illustration of the wireless power transfer system of FIG. 1 being used to supply power to a ventricular assist device (VAD).

FIG. 3 is a front perspective view of one example of a resonator that may be used to implement the system shown in FIG. 1.

FIG. 4 is a perspective view of one example of a resonator assembly that may be used to implement the system shown in FIG. 1.

FIG. 5 is a cross-sectional view of an alternative embodiment of a transmit resonator that may be implemented with the system shown in FIG. 1.

FIG. 6 is a diagram illustrating magnetic field lines through a system including the resonator shown in FIG. 5.

FIG. 7 is a cross-sectional view of an alternative embodiment of a transmit resonator 700 that may be implemented with the system shown in FIG. 1.

### DETAILED DESCRIPTION OF THE DISCLOSURE

The present disclosure is directed to systems and methods for wireless power transfer systems. A resonator arrangement includes a housing, a magnetic core positioned within the housing and defining an annular groove, a coil element positioned within the annular groove and configured to generate a first magnetic field, and a counter-coil element positioned proximate the coil element. The counter-coil element is configured to generate a second magnetic field that is out of phase with the first magnetic field to facilitate reducing far-field electromagnetic emissions.

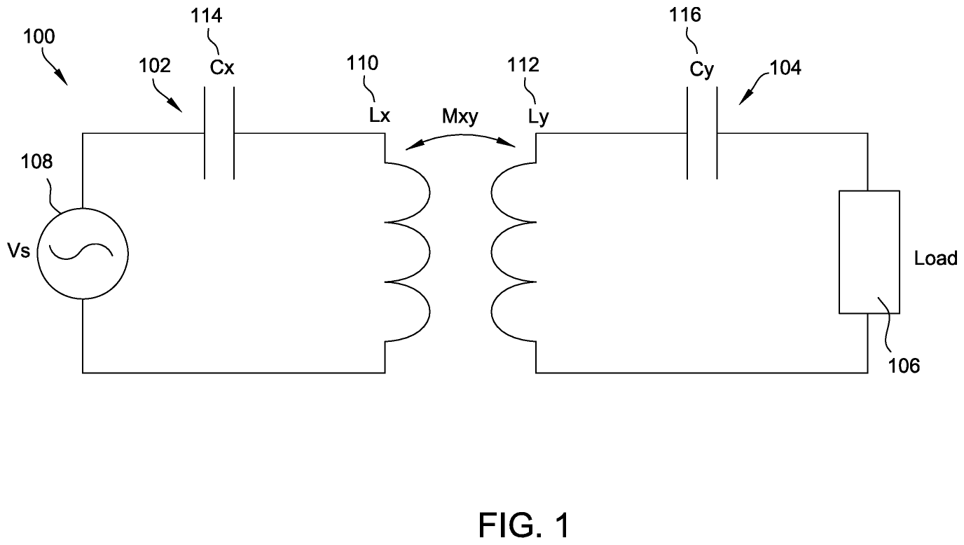

Referring now to the drawings, FIG. 1 illustrates a simplified circuit of a wireless power transfer system 100 according to an example embodiment. The system 100 includes an external transmit resonator 102 and an implantable receive resonator 104. In the system shown in FIG. 1, a power source Vs 108 is electrically connected with the transmit resonator 102, thereby providing power to the transmit resonator 102. The receive resonator 104 is connected to a load 106 (e.g., an implantable medical device). The receive resonator 104 and the load 106 may be electrically connected with a switching or rectifying device (not shown).

In an example, the transmit resonator 102 includes a coil Lx 110 connected to the power source Vs 108 by a capacitor Cx 114. Further, the receive resonator 104 includes a coil Ly 112 connected to the load 106 by a capacitor Cy 116. Inductors Lx 110 and Ly 112 are coupled by a coupling coefficient k. M_{xy} is the mutual inductance between the two coils. The mutual inductance, M_{xy}, is related to the coupling coefficient k as shown in the below Equation (1).

$$\\ \begin{matrix}{M}_{xy} = k \sqrt{{L}_{x} \cdot {L}_{y}} & ( 1 )\end{matrix}$$

In operation, the transmit resonator 102 transmits wireless power received from the power source Vs 108. Receive resonator 104 receives the power wirelessly transmitted by transmit resonator 102 and transmits the received power to load 106.

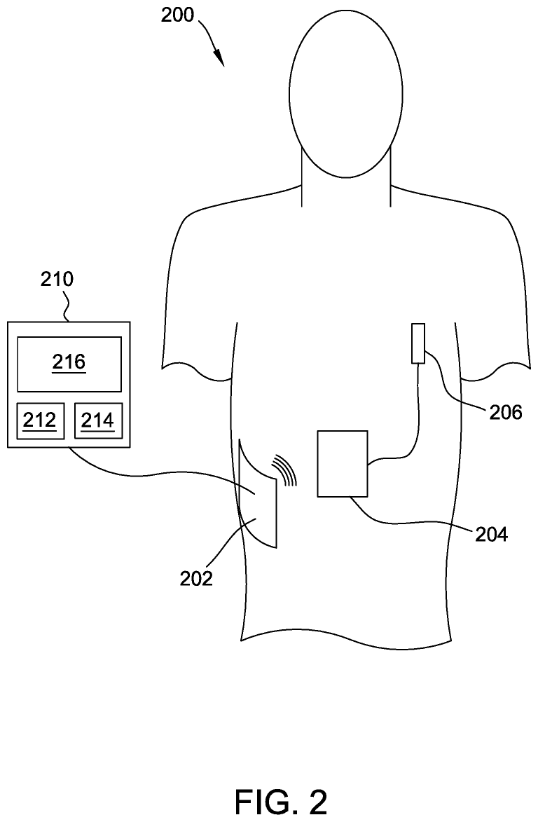

FIG. 2 illustrates an example of a patient 200 using an external coil 202 (e.g., a transmit resonator 102 (FIG. 1)) to wirelessly transmit power to an implanted coil 204 (e.g., a receive resonator 104 (FIG. 1)). Implanted coil 204 uses the received power to power an implanted device 206. For example, implanted device 206 may include a pacemaker or heart pump (e.g., a left ventricular assist device (LVAD)). In some examples, implanted coil 204 and/or implanted device 206 may include or be coupled to a battery.

In one example, external coil 202 is communicatively coupled to a computing device 210, for example, via wired or wireless connection, such that the external coil 202 may receive signals from and transmit signals to the computing device 210. In some examples, the computing device 210 is a power source for the external coil 202. In other examples, the external coil 202 is coupled to an alternative power supply (not shown). The computing device 210 includes a processor 212 in communication with a memory 214. In some examples, executable instructions are stored in the memory 214.

The computing device 210 further includes a user interface (UI) 216. The UI 216 presents information to a user (e.g., the patient 200). For example, the UI 216 may include a display adapter that may be coupled to a display device, such as a cathode ray tube (CRT), a liquid crystal display (LCD), an organic LED (OLED) display, and/or an electronic ink display. In some examples, the UI 216 includes one or more display devices. Further, in some examples, the UI may be or otherwise include a presentation interface. The presentation interface may not generate visual content, but may generate audible and/or computer-generated spoken-word content. In an example, the UI 216 displays one or more representations designed to aid the patient 200 in placing the external coil 202 such that the coupling between the external coil 202 and the implanted coil 204 is optimal. In some examples, the computing device 210 may be a wearable device such as, for example, a wristwatch.

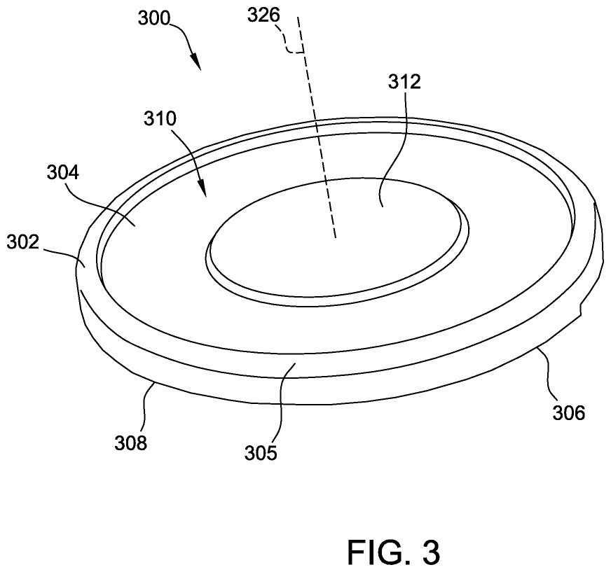

FIG. 3 is a front perspective view of one example of a resonator 300 that may be used to implement the system 100 shown in FIG. 1. For example, the resonator 300 may be used to implement the external transmit resonator 102 (FIG. 1), the implantable receive resonator 104 (FIG. 1), the external coil 202 (FIG. 2), and/or the implanted coil 204 (FIG. 2).

In an example, the resonator 300 includes a core 302 and a coil element 304. The core 302 includes a front surface 305, a back surface 306, and an annular sidewall 308 extending between the front surface 305 and the back surface 306. An annular groove 310 is defined by the front surface 305 and forms a central post 312 of the core 302.

The resonator 300 (including the core 302 and the coil element 304) functions as a wireless power resonator when coupled to a capacitor (e.g., a capacitor on a printed circuit board electrically coupled to coil element 304). However, those of skill in the art will appreciate that resonator 300, without connection to a capacitor, constitutes a coil assembly. Accordingly, as used herein, the term resonator does not require that the device be coupled to a capacitor to form a wireless power resonator. In contrast, as used herein, the term resonator is broad enough to cover a coil assembly that includes a core and a coil element without connection to a capacitor, as shown in FIG. 3.

In an example, the core 302 is formed of a magnetic material. The magnetic material may be a ferrite material, such as nickel-based or manganese-based ferrites. Nickel-based ferrites generally have lower electrical conductivity and reduced losses, while manganese-based ferrites have a higher magnetic permeability (while still having acceptable losses), facilitating containing magnetic field lines, and reducing fringing fields entering nearby conductors (e.g., a titanium enclosure or copper in a nearby PCB) to prevent losses. In other examples, other types of ferrite materials may be used. For example, in some examples, a magnesium-based ferrite (e.g., MgCuZn, which may outperform nickel-based and manganese-based ferrites in a frequency range around 1 Megahertz (MHz)) may be used.

The coil element 304 is positioned within the annular groove 310 and surrounds the central post 312. The resonator 300 may be, for example, a Litz wire resonator or a stacked plate resonator. In a Litz wire resonator, the coil element 304 includes a plurality of loops of Litz wire. In a stacked plate resonator, the coil element 304 includes a plurality of stacked plates that may include a plurality of alternating dielectric layers and conductive layers arranged in a stack. The dielectric layers may be formed of, for example, ceramic, plastic, glass, and/or mica.

The coil element 304 may be electrically coupled to a power source (e.g., when functioning as a transmit resonator) or a load (e.g., when functioning as a receive resonator). In operation, when power is supplied to the resonator 300 operating as a transmit resonator, current flows through the coil element 304, creating an inductive current loop. This inductive current loop is capable of wirelessly transmitting power to a second resonator 300, provided that resonance frequencies of the first and second resonators 300 overlap. The coil element 304 may include a plurality of terminals (not shown) that facilitate electrically coupling the coil element 304 to a power supply or load.

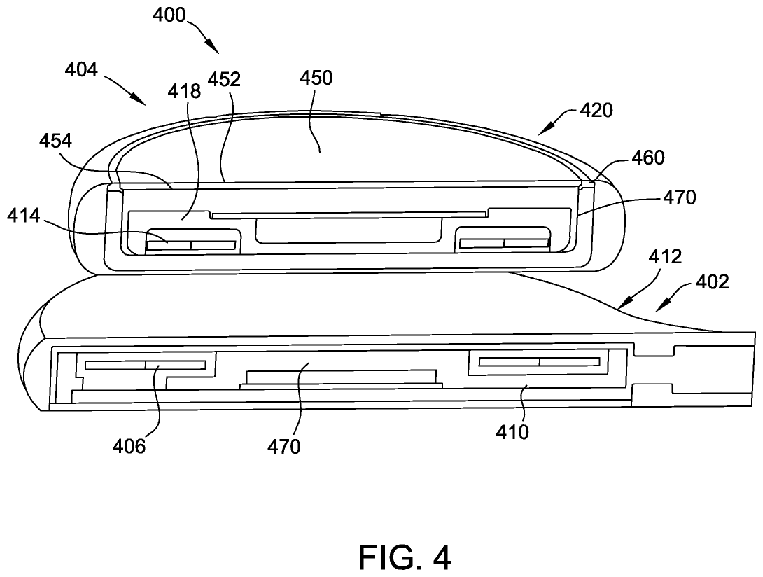

FIG. 4 is a perspective view of a resonator assembly 400. Resonator assembly 400 may include resonators similar to the resonator 300 shown in FIG. 3. The resonator assembly 400 includes a transmit resonator 402 and a receive resonator 404. In an example, the transmit resonator 402 includes a first coil element 406 and a first core 410 positioned within a first housing 412 (e.g., a ceramic housing). Similarly, the receive resonator 404 includes a second coil element 414 and a second core 418 positioned within a second housing 420 (e.g., a ceramic housing). As explained above, the receive resonator 404 is typically implanted within the body, while the transmit resonator 402 is typically external to the body. The transmit and receive resonators 402 and 404 may include one or more electronic components (not shown), such as field-effect transistors (FETs), series inductors, and/or other electronic components.

In this embodiment, the receive resonator 404 further includes a metal disk 450 on a side of the receive resonator 404 opposite the transmit resonator 402. The metal disk 450 may be fabricated from, for example, titanium. The metal disk 450 includes an exterior surface 452 and an interior surface 454 (i.e., that faces the second coil element 414).

In the embodiment of FIG. 4, the receive resonator 404 also includes a metal ring 460 that circumscribes the metal disk 450. The metal ring 460 may be fabricated from the same metal as the metal disk 450 (e.g., titanium). The metal ring 460 may be welded or otherwise coupled to the metal disk 450, and functions as an interface between the metal disk 450 and the second housing 420. Further, the metal ring 460 may be brazed to or otherwise coupled to the second housing 420.

The first core 410 of the transmit resonator 402 is a closed, or solid core. That is, a center 470 of the first core 410 is continuous and contains the same magnetic material as the rest of the first core (such as ferrite). Accordingly, the first core 410 generally has a disk-shape (as opposed to a ring-shape).

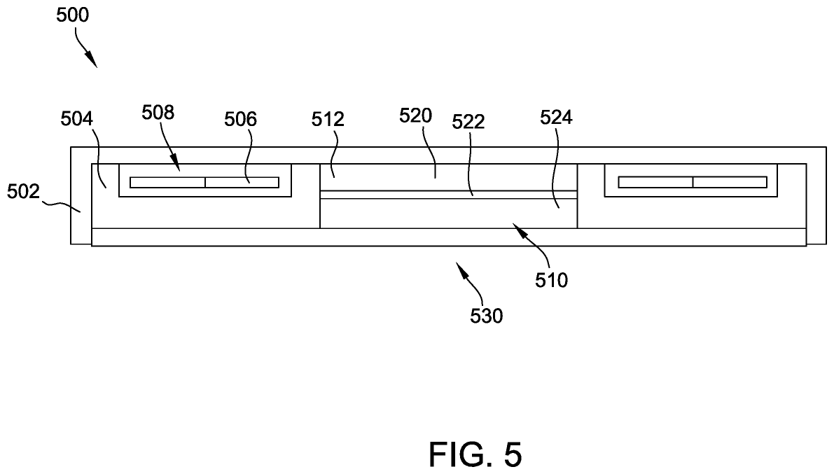

FIG. 5 is a cross-sectional view of an alternative embodiment of a transmit resonator 500 that may be implemented with system 100 (shown in FIG. 1). The resonator 500 includes a housing 502, a core 504, and a coil element 506. The core 504 forms a u-shaped annular groove 508, and the coil element 506 is positioned in the annular groove 508.

As shown in FIG. 5, the core 504 is an open core. That is, the core 504 defines a central aperture 510 that does not contain a metallic or magnetic material. Instead, one or more layers 512 of non-magnetic, non-metallic materials are positioned in the central aperture 510. In the embodiment shown, the layers 512 include a first layer 520, a second layer 522, and a third layer 524. Alternatively, any suitable number of layers 520 may be included. Further, in the embodiment shown, the first, second, and third layers 520, 522, and 524 have different thicknesses. Alternatively, at least some of the first, second, and third layers 520, 522, and 524 may have the same thickness.

The first, second, and third layers 520, 522, and 524 may be made of the same material, or of different materials. Further, the materials used for the first, second, and third layers 520, 522, and 524 may include, for example, aluminum oxide, epoxy (e.g., EpoTek T7110), a printed circuit board (PCB) substrate material, etc. Those of skill in the art will appreciate that any suitable materials may be used. In some embodiments, at least one of the first, second, and third layers 520, 522, and 524 may include a layer of air.

Notably, the open core configuration of the core 504 shown in FIG. 5 results in lower emitted electromagnetic (EM) fields, as compared to a closed core, such as that shown in FIG. 4. Further, depending on the choice of the material(s) in the first, second, and third layers 520, 522, and 524, the overall temperature of the resonator 500 may also be lower, as compared to the transmit resonator shown in FIG. 4.

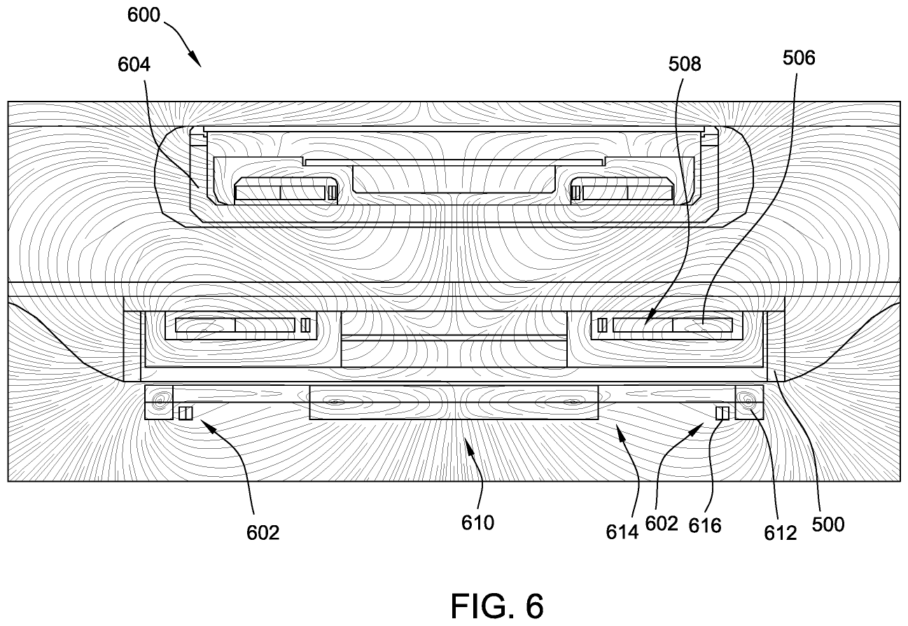

FIG. 6 is a diagram illustrating magnetic field lines 602 through a system 600 including the resonator 500. For clarity, a receive resonator 604 is also shown. Notably, the diagram 600 further includes an additional coil assembly 610 positioned proximate the resonator 500 on a side of the resonator 500 opposite the receive resonator 604 (also referred to as a back side of the resonator 500). In some embodiments, the additional coil assembly 610 may be coupled to the resonator 500.

In this embodiment, the additional coil assembly 610 includes a core 612 defining a u-shaped annular groove 614, and a counter-coil element 616 positioned within the groove 614. The groove 614 faces the opposite direction of the groove 508 in the resonator 500. The counter-coil element 616 may include a plurality of loops of Litz wire, or a plurality of stacked plates, similar to the coil elements described above.

Running a current through the counter-coil element 616 generates a magnetic field that is out of phase with respect to the magnetic field generated by the coil element 506. For example, when the counter-coil element 616 incudes loops of Litz wire, the loops have windings in the opposite direction from a winding of the coil element 506, which results in the counter-coil element 616 operating at a phase that is 180° opposite from the coil element 506. Further, a number of amp-turns in the counter-coil element 616 may be a fraction of a number of amp-turns on the coil element 506, with the fraction equal to a ratio of the number of turns in the counter-coil element 616 divided by the number of turns in the coil element 506.

In another embodiment, a separate set of electronics (e.g., an inverter, etc.) may be used to drive the current through the counter-coil element 616 such that the phase and amp-turns are different from the coil element 506. This implementation allows for controlling the phase and amp-turns of the counter-coil 616 independently.

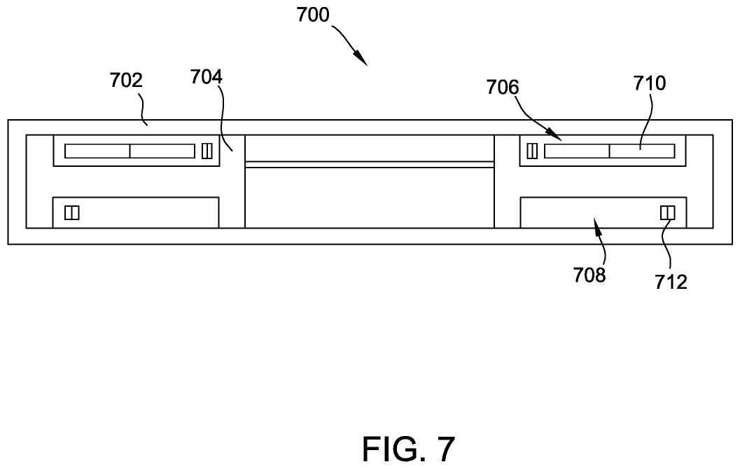

In the embodiment shown in FIG. 6, the core 612 for the counter-coil element 616 is a separate core from the core 504 for the coil element 506. In some embodiments, the counter-coil element 616 and the coil element 506 share a core (see, e.g., FIG. 7). Further, in some embodiments, an additional counter-coil element (not shown) may be included, to facilitate further reducing EM emissions.

As shown in FIG. 6, the counter-coil element 616 generates a magnetic field 620 that at least partially cancels out the far-field EM emissions generated by the coil element 506. As used herein, far-field EM emissions refer to EM emissions at a distance from the coil element 506 that is greater than an outer diameter of the core 504 (including distance several times larger than the outer diameter of the core 504).

For example, in one embodiment, far-field EM emissions may be reduced by up to 32.5% in a region directly behind the resonator 500 (i.e., on a side opposite the receive resonator 604). Even further reduction of EM emissions is possible when the phase and amp-turns of the counter-coil 616 are controlled independently (e.g., a reduction of up to 60%, or 7.9 dB).

In some embodiments, EM emissions emanate from two physically distinct coils in the resonator 500—an exciter coil and a resonator coil. Although the resonator coil is the primary source of emissions, both contribute. Further the exciter coil and the resonator coil do not operate in phase with one another. Accordingly, in such embodiments, the counter-coil element 616 may be operated at a phase that is 180° opposite from a weighted average of the phases of the exciter coil and the resonator coil.

FIG. 7 is a cross-sectional view of an alternative embodiment of a transmit resonator 700 that may be implemented with system 100 (shown in FIG. 1). The resonator 700 includes a housing 702 and a core 704. In this embodiment, the core 704 defines a first u-shaped annular groove 706 and a second u-shaped annular groove 708 that face in opposite directions. A transmitter coil element 710 is positioned in the first u-shaped annular groove 706, and a counter-coil element 712 is positioned in the second u-shaped annular groover 708. That is, in this embodiment, the transmitter coil element 710 and the counter-coil element 712 are positioned on opposite sides of the same core 704 (and are both positioned within a housing 702 of the resonator 700. Having the transmitter coil element 710 and the counter-coil element 712 share the same core 704 reduces the additional space and weight required to include the counter-coil element 712.

Notably, while the counter-coil elements described significantly decrease far-field EM emissions behind the transmit resonator, EM emissions elsewhere (e.g., behind the receive resonator) are minimally impacted.

The embodiments described herein are directed to systems and methods for wireless power transfer systems. A resonator arrangement includes a housing, a magnetic core positioned within the housing and defining an annular groove, a coil element positioned within the annular groove and configured to generate a first magnetic field, and a counter-coil element positioned proximate the coil element. The counter-coil element is configured to generate a second magnetic field that is out of phase with the first magnetic field to facilitate reducing far-field electromagnetic emissions.

Although the embodiments and examples disclosed herein have been described with reference to particular embodiments, it is to be understood that these embodiments and examples are merely illustrative of the principles and applications of the present disclosure. It is therefore to be understood that numerous modifications can be made to the illustrative embodiments and examples and that other arrangements can be devised without departing from the spirit and scope of the present disclosure as defined by the claims. Thus, it is intended that the present application cover the modifications and variations of these embodiments and their equivalents.

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A resonator arrangement for use in a wireless power transfer system, the resonator arrangement comprising:
a housing;
a magnetic core positioned within the housing and defining an annular groove;
a coil element positioned within the annular groove and configured to generate a first magnetic field; and
a counter-coil element positioned proximate the coil element, the counter-coil element configured to generate a second magnetic field that is out of phase with the first magnetic field to facilitate reducing far-field electromagnetic emissions.

2. The resonator arrangement of claim 1, wherein the counter-coil element is positioned outside of the housing.

3. The resonator arrangement of claim 2, wherein the magnetic core is a first magnetic core defining a first annular groove, and wherein the resonator arrangement further comprises a second magnetic core, the counter-coil element positioned within a second annular groove defined by the second magnetic core.

4. The resonator arrangement of claim 1, wherein the counter-coil element is positioned within the housing.

5. The resonator arrangement of claim 4, wherein the magnetic core further defines an additional annular groove, the counter-coil element positioned within the additional annular groove.

6. The resonator arrangement of claim 1, wherein the coil element is a transmit coil element, and wherein the first magnetic field is operable to induce a current in a receive coil element.

7. The resonator arrangement of claim 6, wherein the counter-coil element is positioned on a side of the transmit coil element opposite the receive coil element.

8. A transcutaneous energy transfer system (TETS) comprising:
a transmit resonator comprising:
a first housing;
a first magnetic core defining a first annular groove; and
a transmit coil element positioned within the first annular groove and configured to generate a first magnetic field:
an implantable receive resonator comprising:
a second housing;
a second magnetic core defining a second annular groove; and
a receive coil element positioned within the second annular groove, wherein the first magnetic field is configured to induce a current in the receive coil element; and
a counter-coil element positioned proximate the transmit coil element, the counter-coil element configured to generate a second magnetic field that is out of phase with the first magnetic field to facilitate reducing far-field electromagnetic emissions.

9. The TETS of claim 8, wherein the counter-coil element is positioned outside of the first housing.

10. The TETS of claim 9, further comprising a third magnetic core defining a third annular groove, the counter-coil element positioned within the third annular groove.

11. The TETS of claim 10, wherein the third magnetic core is coupled to the first housing.

12. The TETS of claim 8, wherein the counter-coil element is positioned within the first housing.

13. The TETS of claim 12, wherein the first magnetic core further defines a third annular groove, the counter-coil element positioned within the third annular groove.

14. The TETS of claim 13, wherein the first annular groove and the third annular groove face in opposite directions.

15. A method of assembling a resonator arrangement for use in a wireless power transfer system, the method comprising:
positioning a magnetic core within a housing, the magnetic core defining an annular groove;
positioning a coil element within the annular groove, the coil element configured to generate a first magnetic field; and
positioning a counter-coil element proximate the coil element, the counter-coil element configured to generate a second magnetic field that is out of phase with the first magnetic field to facilitate reducing far-field electromagnetic emissions.

16. The method of claim 15, wherein positioning the counter-coil element comprises positioning the counter-coil element outside of the housing.

17. The method of claim 16, wherein the magnetic core is a first magnetic core defining a first annular groove, and wherein the method further comprises positioning the counter-coil element within a second annular groove defined by a second magnetic core.

18. The method of claim 17, further comprising coupling the second magnetic core to the housing.

19. The method of claim 15, wherein positioning the counter-coil element comprises positioning the counter-coil element within the housing.

20. The method of claim 19, wherein positioning the counter-coil element within the housing comprises positioning the counter-coil element within an additional groove defined by the magnetic core.
